(19) United States
(12) Patent Application Publication
(10) Pub. No.: US 2024/0279263 A1
(43) Pub. Date: Aug. 22, 2024

(54) NOVEL SEA ALGAE-DERIVED ALKYL-AGARBIOSIDE, PREPARATION METHOD THEREFOR, OR USE THEREOF

(71) Applicant: KOREA UNIVERSITY RESEARCH AND BUSINESS FOUNDATION, Seoul (KR)

(72) Inventors: Kyoung Heon KIM, Seoul (KR); Eun Ju YUN, Seoul (KR); Sun Hee LEE, Seoul (KR); Nam Joo KANG, Daegu (KR)

(73) Assignee: KOREA UNIVERSITY RESEARCH AND BUSINESS FOUNDATION, Seoul (KR)

(21) Appl. No.: 18/289,972

(22) PCT Filed: May 9, 2022
(86) PCT No.: PCT/KR2022/006570

§ 371 Date: Dec. 14, 2023

(30) Foreign Application Priority Data
May 10, 2021 (KR) 10-2021-0059886

(51) Int. Cl. C07H 3/10; A61K 8/49; C07H 1/06

(52) U.S. Cl. CPC C07H 3/10; A61K 8/498; C07H 1/06

(57) ABSTRACT

The present invention identifies moisturizing and physiological activities of agarobiose and provides a novel substance alkyl-agarobioside for maintaining moisturization activity of agarbiose and securing both pH and temperature stability, and a preparation method therefor. According to the present invention, alkyl-agarobioside can be produced in a cost-efficient manner as a moisturizing material for foods, medicinal products, and cosmetic products.

### TECHNICAL FIELD

The present invention relates to alkyl-agarobioside, which is agarobioside derived from sea algae, a production method thereof, or a use thereof.

### BACKGROUND ART

The main polysaccharide constituting red algae is agarose, which is a polymer of alternating 3,6-anhydro-L-galactose (hereinafter referred to as “AHG”) and D-galactose linked by α-(1,3) and β-(1,4) bonds (Chi, W.-J et al (2012) Applied Microbiology and Biotechnology. 94(4): 917-930; Yun, E. J et al (2017) Applied Microbiology and Biotechnology. 101(14): 5581-558).

Among them, L-AHG is a rare sugar that is not found in terrestrial organisms and can be used as a material for cosmetic products due to its excellent whitening and moisturizing effects, and it is a multi-functional high value-added material with anti-inflammatory, anti-cavity, and colon cancer prevention effects. Due to such functionality, studies on red algae have been conducted to produce L-AHG (Kim, D. H (2018) Journal of Agricultural and Food Chemistry. 66(46): 12249-12256).

In addition, agarobiose (hereinafter referred to as “AB”), a disaccharide having L-AHG at its reducing end, is known to have an excellent anti-cavity effect (Sora Yu et al (2019) Journal of Agricultural and Food Chemistry. 67: 7297-7393).

Despite L-AHG being a multi-functional high value-added material, it is difficult to use L-AHG for industrial purposes. This is because L-AHG is very unstable at high temperatures and acid conditions, so L-AHG is easily converted to 5-hydroxymethylfurfural (5-HMF) and loses its functionality (Yang, B et al (2009) The FEBS Journal. 276(7): 2125-2137; Jeong, G.-T (2015) Bioprocess and Biosystems Engineering. 38(2): 207-217).

In order to solve this problem, the present inventors have established optimal conditions to produce high concentration agarobiose (AB) through acid hydrolysis and neutralization of agarose or agar in previous research (Korean Registered Patent No. 10-1864800). However, studies on the efficacy and stability of agarobiose have never been conducted.

### Related Art Documents

### Patent Documents

Korean Registered Patent No. 10-1864800

### DISCLOSURE

### Technical Problem

The present inventors intended to confirm a new physiological activity of agarobiose and provide a method capable of ensuring stability while maintaining the physiological activity of agarobiose and a new compound produced therefrom.

The present invention is directed to providing alkyl-agarobioside, a derivative thereof, or a salt thereof.

The present invention is also directed to providing a method of producing alkyl-agarobioside.

The present invention is also directed to providing a composition including agarobiose or alkyl-agarobioside as an active ingredient, specifically a moisturizing cosmetic composition.

### Technical Solution

The present inventors found that agarobiose produced according to the agarobiose production method of the related art (Korean Registered Patent No. 10-1864800) has a moisturizing effect on HaCaT cells, which are skin cells, but it was confirmed that agarobiose was decomposed within 6 weeks in basic conditions (pH 9) unlike acidic or neutral conditions, causing stability problems when agarobiose is formulated into cosmetic products or applied to the human body.

To solve this problem, the present inventors produced alkyl-agarobioside by introducing an alkyl group into agarobiose and confirmed that the produced alkyl-agarobioside exhibits the same moisturizing activity as agarobiose and has better stability in various pH conditions compared to agarobiose, and thereby completed the present invention.

One aspect of the present invention provides a compound represented by Chemical Formula 1 below, a derivative thereof, or a salt thereof:

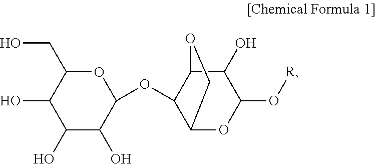

in Chemical Formula 1,
R may be a C₁₋₅ alkyl group, preferably a C₁₋₃ alkyl group, more preferably an ethyl group.

In the present invention, the compound represented by Chemical Formula 1 may be referred to as alkyl-agarobioside, and in a specific example, the compound in which R is an ethyl group may be referred to as ethyl-agarobioside (ethyl-AB).

Another aspect of the present invention provides a method of producing a compound represented by Chemical Formula 1 below:

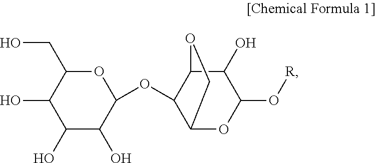

in Chemical Formula 1,
R may be a C₁₋₅ alkyl group, preferably a C₁₋₃ alkyl group, and more preferably an ethyl group.

Specifically, the method may include: treating agar or agarose with a strong acid and an alkanol; neutralizing a product from the treating process; and separating and purifying the compound represented by Chemical Formula 1 from the neutralized product.

Specifically, alkyl-agarobioside may be produced by reacting 1 or 20% (w/v) of agar or agarose with a 1 to 100 mM strong acid and alkanol to induce acid-catalyzed alcoholysis in one step.

More specifically, the strong acid may be one or more of hydrochloric acid, nitric acid, and sulfuric acid, and preferably, when sulfuric acid is used, a yield of alkyl-agarobioside may be further increased. Additionally, a strong acid concentration of 1 to 100 mM, more specifically, 7 to 70 mM may be used in treating agar or agarose with the strong acid. In this concentration range, high concentration alkyl-agarobioside may be produced from agar or agarose through acid-catalyzed alcoholysis.

An amount of agar or agarose, which is used in acid-catalyzed alcoholysis as a substrate, may be 1 to 20% (w/v), more specifically 1 to 5% (w/v), still more specifically 2 (w/w), based on dry weight. In this range, a liquefaction rate may be 90%, 95%, or 98% or more. When outside this range, a decomposition rate of the substrate may significantly decrease.

The acid-catalyzed alcoholysis may be carried out at 50 to 140° C. for 12 to 24 hours. Specifically, the acid-catalyzed alcoholysis may be carried out at 60 to 100° C. for 16 to 20 hours.

The neutralizing process is adding a strong base to a product that underwent acid-catalyzed alcoholysis and neutralizing the product to have a pH level of 5 to 7. The strong base may be NaOH, KOH, Ca(OH)₂, or Ba(OH)₂ but is not limited thereto.

The above-described method is designed to induce alcoholysis based on the optimized method of producing high concentration agarobiose from agarose as proven in the related art (Korean Registered Patent No. 10-1864800), and therefore, high concentration alkyl-agarobioside may be produced from agarose using the advantages of the related art.

Still another aspect of the present invention provides a skin moisturizing composition including, as an active ingredient, the alkyl-agarobioside compound according to the present invention, a derivative thereof, or a salt thereof.

In particular, the present invention is not limited to producing the novel agarobioside compound and providing a production method thereof, but also confirms the skin moisturizing efficacy and effectiveness of agarobiose or the alkyl-agarobioside produced according to the present invention for cosmetic use and produces a cosmetic composition. Therefore, regardless of whether the substance disclosed in the claims is a novel substance, the discovery that the substance can be used as cosmetic products, such as a skin moisturizer, should have great technical significance.

In particular, it was confirmed that alkyl-agarobioside produced by the production method of the present invention exhibits the same skin moisturizing activity as agarobiose and has excellent pH stability. In a specific example, as shown in FIG. 8, it was confirmed that the produced ethyl-agarobioside maintains stability under various temperature conditions (4° C., 30° C., or 45° C.). However, unlike other temperature conditions, results of component analysis at high temperature of 45° C. or higher showed that ethyl-agarobioside and agarobiose are both present, indicating that ethyl-agarobioside is converted to agarobiose and remains in the agarobiose state without further thermal conversion (FIG. 9). According to these results, as ethyl-agarobioside is converted to agarobiose with excellent temperature stability while exhibiting the same moisturizing activity at high temperatures as well as excellent pH stability, the composition including ethyl-agarobioside as an active ingredient can have high temperature stability without affecting moisturizing activity. A more comprehensive understanding of this can be obtained through examples and experimental examples that will be described later.

In the present invention, “moisturizing or skin moisturizing” refers to increasing moisture in the skin and maintaining moisture in the skin. The skin moisturizing effect may help reduce skin wrinkles and improve skin elasticity.

In the cosmetic composition according to the present invention, a content of the compound, a derivative thereof, or a salt thereof is preferably 0.00001 to 10 wt % based on a total weight of the cosmetic composition.

The “salt” of the present invention preferably includes an acid or base addition salt, wherein the base addition salt includes, but is not limited to, a salt consisting of sodium, potassium, calcium, ammonium, magnesium, or an organic amino.

The compound of the present invention may be present not only in solvated forms including hydrates, ethanolates, and the like but also in unsolvated forms. The alkyl-agarobioside of the present invention may be present in a crystalline or amorphous form, and all such physical forms are included in the scope of the present invention.

The skin moisturizing cosmetic composition according to the present invention may be prepared in a formulation selected from the group consisting of a solution, an ointment for external use, a cream, a foam, a nourishing lotion, a softening lotion, a facial mask, a softener, an emulsion, a makeup base, an essence, a soap, a liquid cleanser, a bath salt, a sunscreen cream, a tanning oil, a suspension, an oil emulsion, a paste, a gel, a lotion, a powder, a surfactant-containing cleanser, an oil, a powder foundation, an emulsion foundation, a wax foundation, a patch, and a spray, but is not limited thereto.

The cosmetic composition of the present invention may further include one or more cosmetically acceptable carriers mixed with commonly used skin cosmetics, and common ingredients such as oil, water, a surfactant, a moisturizer, a lower alcohol, a thickener, a chelating agent, a pigment, a preservative, a fragrance, and the like may be appropriately mixed, but are not limited thereto.

### Advantageous Effects

The present invention confirms the physiological moisturizing activity of agarobiose and provides alkyl-agarobioside, which is a novel substance, to maintain the moisturizing activity of agarobiose and ensure pH stability and temperature stability, and a production method thereof, and therefore, alkyl-agarobioside can be cost-effectively produced as an excellent moisturizing material for food, medicines, and cosmetics.

### DESCRIPTION OF DRAWINGS

FIG. 1 illustrates the results of testing the HAS2 expression effect of AB in HaCaT cells, which are human skin cells, and the pH stability of AB: (A) cytotoxicity according to AB concentration, (B) HAS2 expression level in HaCaT cells according to AB concentration, and (C) pH stability of AB.

FIG. 2 illustrates the results of measuring the produced ethyl-agarobioside according to the sulfuric acid concentration: (A) the produced ethyl-agarobioside concentration according to the sulfuric acid concentration, and (B) HPLC results of the produced ethyl-agarobioside according to the sulfuric acid concentration.

FIG. 3 illustrates the HPLC results of the produced ethyl-agarobioside according to the type of strong acid.

FIG. 4 illustrates the results of measuring ethyl-agarobioside purified using size exclusion chromatograph with G-10 resin: (A) the product obtained by decomposing agarose with sulfuric acid and ethanol, and (B) the result of separation and purification using size exclusion chromatography with G-10 resin.

FIG. 5 illustrates the LC-HRMS and 2D-HSQC NMR analyses results of the separated and purified ethyl-agarobioside: (A) LC-HRMS analysis results of separated and purified ethyl-agarobioside, and (B) 2D-HSQC NMR analysis results of separated and purified ethyl-agarobioside.

FIG. 6 illustrates the results of confirming the HAS2 expression effect of ethyl-agarobioside in HaCaT cells, which are human skin cells, and the pH stability of ethyl-agarobioside: (A) cytotoxicity according to ethyl-agarobioside concentration, (B) HAS2 expression level in HaCaT cells according to ethyl-agarobioside concentration, and (C) pH stability of ethyl-agarobioside.

FIG. 7 illustrates the results of comparing HAS2 expression levels when HaCaT cells, which are human skin cells, are treated with 100 μg/mL of L-AHG, AB, and ethyl-agarobioside.

FIG. 8 illustrates the results of comparing the temperature stability of AB and ethyl-agarobioside.

FIG. 9 illustrates the comparison of concentration changes of AB and ethyl-agarobioside by temperature to confirm the temperature stability of ethyl-agarobioside: (A) 4° ° C., (B) 30° C., and (C) 45° C.

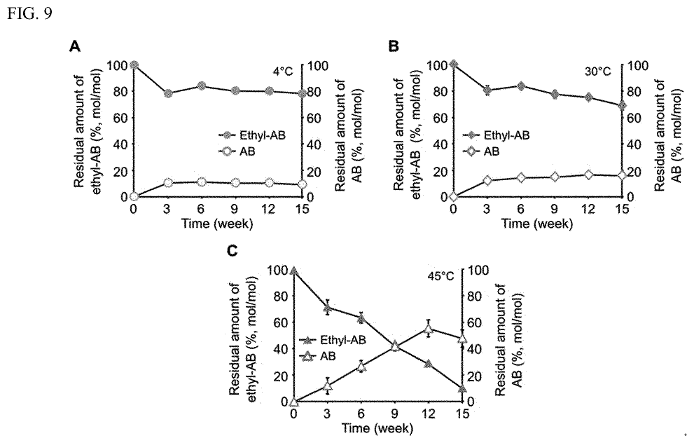

### MODES OF THE INVENTION

Hereinafter, the present invention will be described in detail by examples. However, the following examples are given for the purpose of illustration only, and the present invention is not limited to the examples described below.

### [Example 1] Moisturizing Effect of AB on HaCaT Cells (Human Skin Cells)

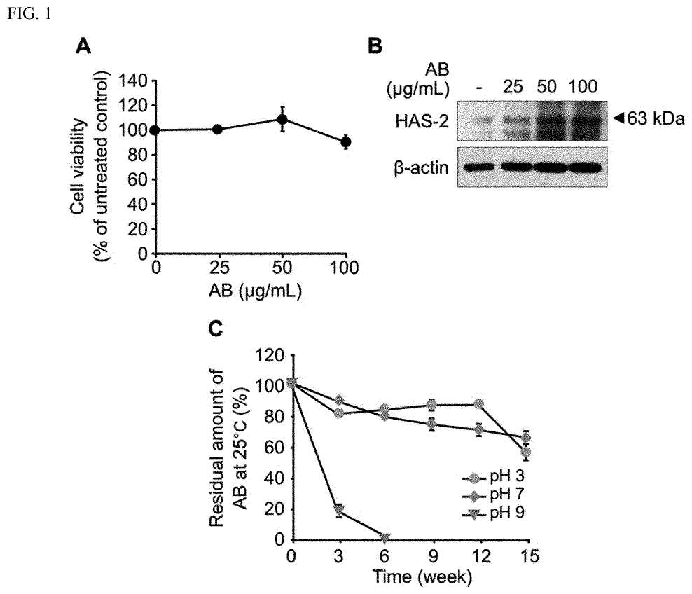

AB was produced from sea algae according to the related art (Korean Registered Patent No. 10-1864800), and the produced AB was confirmed to have a moisturizing effect in HaCaT cells, which are human skin cells, and pH stability (FIG. 1).

Cytotoxicity tests were performed using the MTT(3-4,5-dimethylthiazol-2yl)-2,5-diphenyl-2H-tetrazolium bromide) assay. HaCaT cells were cultured in an animal cell incubator at 37° C. and 5% CO₂ in a laboratory using Dulbecco's Modified Eagle Medium (DMEM) containing 10% (v/v) FBS, 100 U/mL penicillin, and 100 g/mL streptomycin as antibiotics. The produced AB was dissolved in DMSO and added to the culture medium at 0-100 g/mL, and cells were cultured in the medium for 24 hours. The cells were treated with an MTT solution (5 mg/mL) and cultured for another 4 hours, and then an amount of the produced formazan as a blue crystal, was measured by measuring absorbance at 595 nm using an ELISA reader. Toxicity to cells was expressed as a percentage of the average absorbance value of each control group. To investigate the cytotoxicity of AB in HaCaT keratinocytes, when AB was treated at a concentration of 0-100 g/mL, the degree of change in cell proliferation was calculated compared to control groups treated with only DMSO. The results of Example 1 are shown in A of FIG. 1.

B of FIG. 1 illustrates the presence or absence of a cytotoxic effect when HaCaT keratinocytes are treated with AB according to an example of the present invention. Cell viability was measured to be 100% or more at 100 g/mL AB. Therefore, it was confirmed that there is no cytotoxicity below 100 g/mL AB.

Hyaluronan (HA) is a glycosaminoglycan composed of D-glucuronic acid and N-acetyl-D-glucosamine. Due to its ability to retain large amounts of water, HA plays an important role in regulating hydration and osmotic pressure. AH is synthesized in cell membranes by HAS1, HAS2, and HAS3, and HAS2 in particular appears in human normal tissues. In previous studies, it was found that a genetic defect of HAS2 causes fetal lethality in a mouse model and shows reduced HAS2 gene expression in the epidermis and dermis of adult human skin. Therefore, increasing HAS2 expression may be a great strategy to maintain skin homeostasis. To determine the AB induction time and AB concentration for HAS2 expression, Western blot analysis was performed. Cells used for the analysis were HaCaT cells, which were cultured at 37° C. in a 5% CO₂ atmosphere using DMEM with 10% FBS and penicillin/streptomycin. Cells (1×10⁵) were cultured in 6-cm dishes for 24 hours and then starved in serum-free medium for another 24 hours to remove the FBS effect on the activation of the kinase. Then, AB was treated at a certain time and concentration. Cells were lysed with a lysis buffer [20 mM Tris-HCl (pH 7.5), 150 mM NaCl, 1 mM Na₂EDTA, 1 mM EGTA, 1% Triton X-100, 2.5 mM sodium pyrophosphate, 1 mM β-glycerophosphate, 1 mM Na₃VO₄, 1 g/mL leupeptin, 1 mM phenylmethylsulfonyl fluoride (PMSF), and a protease inhibitor cocktail tablet]. The protein concentration was measured using a dye-conjugated protein assay kit (Bio-Rad Laboratories Inc.) according to the manufacturer's instructions. Dissolved proteins (20-40 μg) were subjected to 10% SDS-PAGE and transferred to a polyvinylidene fluoride (PVDF) membrane by electrophoresis (Millipore Corp., Bedford, MA, USA). After blotting, the membrane was blocked with 5% skim milk for 2 hours and incubated overnight at 4° C. with a primary antibody (goat anti-mouse IgG-HRP). Subsequently, the membrane was incubated with a secondary antibody (goat anti-rabbit IgG HRP-conjugated secondary antibody), and the protein bound to the antibody was detected using a chemiluminescence detection kit (Amersham Pharmacia Biotech, Piscataway, NJ). Representative values of two independent experiments were used as data. As shown in B of FIG. 1, AB increased HAS2 expression 3 hours after treatment. In addition, AB increased HAS2 expression in a concentration-dependent manner. The moisturizing activity of AB was confirmed from the result.

### [Example 2] pH Stability of AB

The pH stability of AB was assessed (C of FIG. 1). For a pH stability experiment, the degree of denaturation in each sample (AB) over time was measured at 25° C. under conditions of pH 3 (20 mM citrate), pH 7 (20 mM Tris-HCl), and pH 9 (20 mM Tris-HCl). As shown in C of FIG. 1, the sample was stable over time at pH 3 and pH 7, but it was found that the sample was completely denatured within 6 weeks at pH 9. Since the buffer and AB peaks were not separated at pH 3 using HPLC, AB was quantified through GC/MS analysis. The derivatization process for GC/MS analysis is as follows. After drying a purified sample with a speed vac, 10 μL of 40 mg/mL O-methylhydroxylamine hydrochloride in pyridine was added and reacted at 30° ° C. and 200 rpm for 90 minutes. Then, 45 μL of N-methyl-N-(trimethylsilyl)trifluoroacetamide was added and reacted at 37° C. and 200 rpm for 30 minutes. Instrument conditions for GC/MS analysis are as follows. A DB5-MS capillary column was used in the analysis, and the GC column temperature condition was maintained at 50° C. for 1 minute and then raised to 280° C. and maintained for 5 minutes. 1 μL of sample was analyzed in a splitless mode. Each concentration of agarose at pH 7 and pH 9 was analyzed using HPLC. HPX-87H was used as a column, and the sample was analyzed at a column temperature of 65° C. and a flow rate of 0.5 mL/min. 0.005 M sulfuric acid was used as a mobile phase.

### [Example 3] Production of Ethyl-Agarobioside from Red Algae (Agarose)

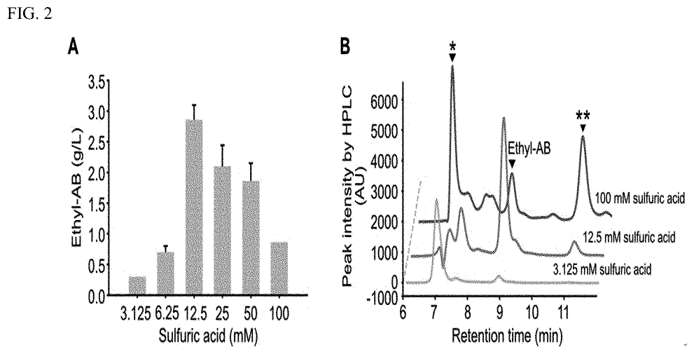

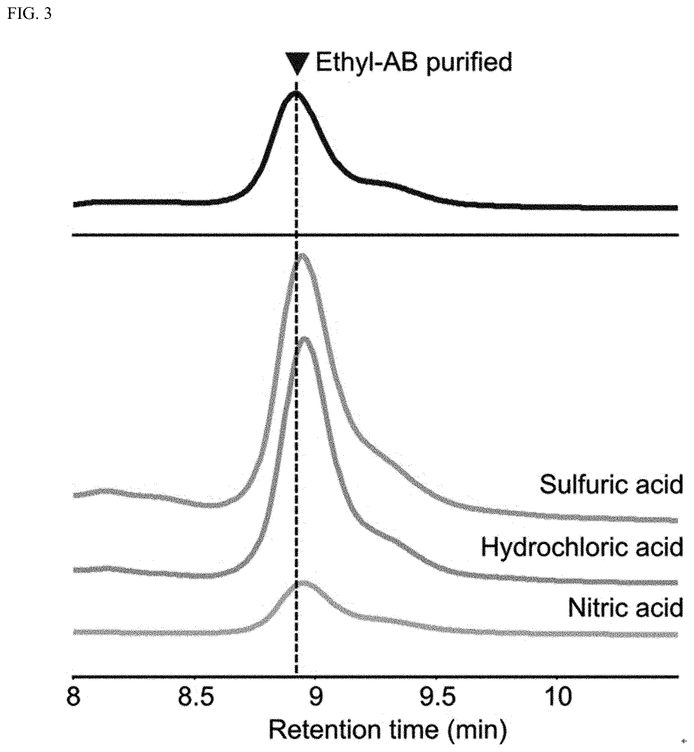

Agarose, which is a representative polysaccharide constituting sea algae, was decomposed using strong acids such as sulfuric acid, hydrochloric acid, and nitric acid. Ethyl-agarobioside was produced through a one-pot reaction of 2% (w/v) agarose with 12.5 mM sulfuric acid and 100 mL of ethanol at 70° C. overnight (about 18 hours). The sulfuric acid was neutralized and removed using tertiary distilled water and calcium hydroxide (Ca(OH)₂) in order to use the ethyl-agarobioside. In addition, to determine the concentration of sulfuric acid that can produce a large amount of ethyl-agarobioside, experiments were conducted with 3.125 mM, 6.25 mM, 12.5 mM, 25 mM, 50 mM, and 100 mM of sulfuric acid, and the produced ethyl-agarobioside was quantified, and it was confirmed that the greatest amount of ethyl-agarobioside was produced with 12.5 mM sulfuric acid (FIG. 2). In addition, it was confirmed that when agarose was treated with hydrochloric acid (12.5 mM) or nitric acid (12.5 mM, 25 mM) in a 20 mL reaction volume under the same experimental conditions as sulfuric acid above, the greatest amount of ethyl-agarobioside was produced in each case (FIG. 3).

### [Example 4] Separation and Purification of Ethyl-Agarobioside from Fermentation Products Using Size-Exclusion Chromatography

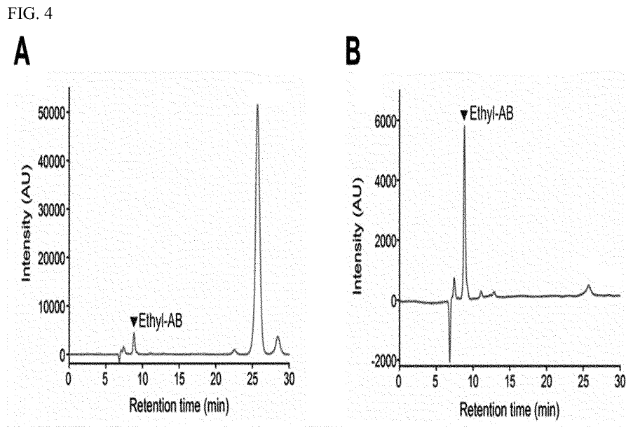

Size-exclusion chromatography was used to separate and purify ethyl-agarobioside produced in Example 3. Sephadex G-10 (GE Healthcare) was used as a resin, and distilled water was used as a mobile phase (FIG. 4).

### [Example 5] Identification of Molecular Weight and Structure of Ethyl-Agarobioside Through LC-HRMS and 2D HSQC NMR Analyses

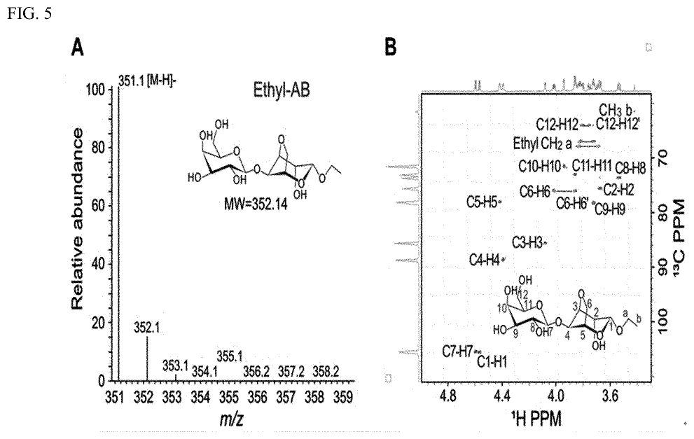

LC-HRMS and 2D HSQC NMR analyses were conducted to identify the molecular weight and chemical structure of ethyl-agarobioside produced in Examples 3 and 4 (FIG. 5). LC-HRMS analysis results showed that the molecular weight of ethyl-agarobioside was 352.14 when agarose was converted to ethyl-agarobioside. Excluding a hydrogen ion, the actual molecular weight measured became 351. 2 mg of an ethyl-agarobioside sample was dissolved in D₂O, and 3-(trimethylsilyl)-propionic-2,2,3,3-d4 acid was used as an internal standard to calculate a chemical shift. Chemical shifts in 2D HSQC NMR analysis were compared to previously reported results in documents to identify the chemical structure (Anatoly I. Usov et al (1980) Biopolymers, 19: 977-990; Cyrille Rochas (1986) Carbohydrate Research, 148: 199-207; E. Murano (1992) Carbohydrate Polymers. 18: 171-178).

### [Example 6] Experiment for Moisturizing Effect of Ethyl-Agarobioside in HaCaT Cells (Human Skin Cells)

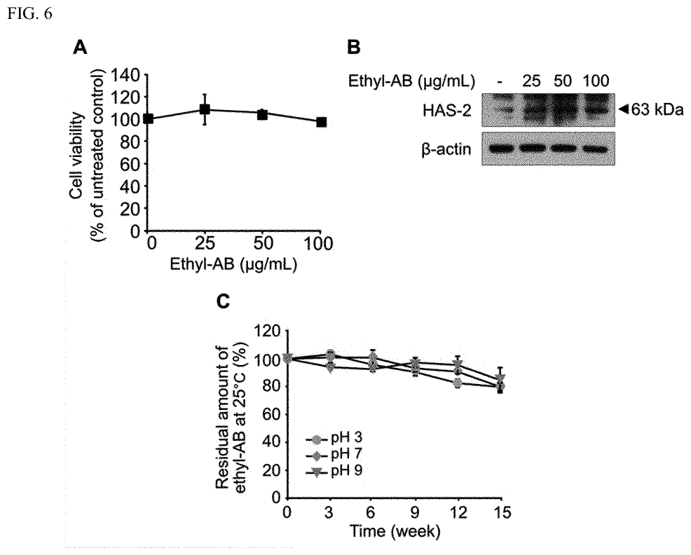

The experiment was performed in the same manner as Example 1, and it was confirmed that ethyl-agarobioside also exhibits moisturizing activity without cytotoxicity in HaCaT cells, which are human skin cells (A of FIG. 6 and B of FIG. 6).

### [Example 7] pH Stability of Ethyl-Agarobioside

The experiment was performed in the same manner as Example 2, and it was confirmed that the stability of ethyl-agarobioside was maintained at pH 3, pH 7, and pH 9. The ethyl-agarobioside concentration was measured using GC-MS and HPLC (C of FIG. 6).

### [Example 8] Experiment for Comparing HAS2 Expression of L-AHG, AB, and Ethyl-Agarobioside in HaCaT Cells (Human Skin Cells)

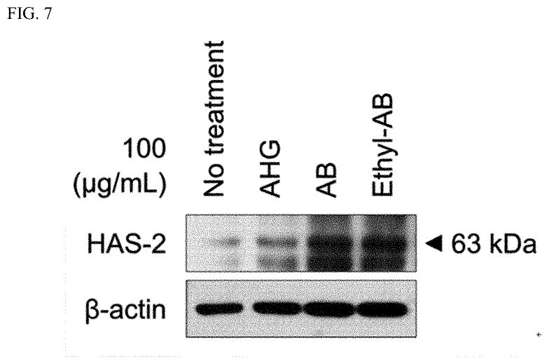

The experiment was performed in the same manner as Example 1. In a previous study, it was reported that L-AHG (Korean Registered Patent No. 10-1525298) has moisturizing activity in HaCaT cells, which are human skin cells, and in the present invention, the effects on the regulation of HAS2 expression in HaCaT cells, which are human skin cells, when AB and ethyl-agarobioside were treated at the same concentration were compared with L-AHG. It was confirmed that, compared to L-AHG showing a moisturizing effect, HAS2 expression was greatly increased when treated with AB and ethyl-agarobioside, so it was expected that the moisturizing effect of AB and ethyl-agarobioside would be better (FIG. 7).

### [Example 9] Temperature Stability of AB and Ethyl-Agarobioside

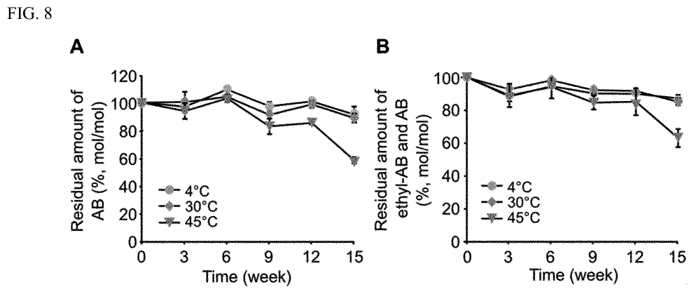

The temperature stability of agarobiose was tested. The experimental method measured the denaturation/decomposition degrees of AB and ethyl-agarobioside over time at 4° C., 30° C., and 45° C. As shown in FIG. 8, it was confirmed that AB maintained stability for 15 weeks at 4° C. and 30° C., but at 45° C., stability slowly decreased. On the other hand, it was confirmed that ethyl-agarobioside maintained stability for 15 weeks at 4° C. and 30° C., but ethyl-agarobioside was less stable than AB after 6 weeks at 45° C. AB and ethyl-agarobioside were quantified using HPLC. As a result of analyzing reactant components of ethyl-agarobioside at 45° C. using HPLC, it was confirmed that AB, which was not present initially, was produced, and as the temperature increased, ethyl-agarobioside was converted to AB and was present along with AB. From these results, it was determined that not only the functionality of the cosmetic composition including ethyl-agarobioside may not be affected because ethyl-agarobioside is converted to AB, exhibiting the same moisturizing activity as ethyl-agarobioside, at high temperatures above 45° C., but also high-temperature stability may be secured.

1. A compound represented by Chemical Formula 1 below, a derivative thereof, or a salt thereof: 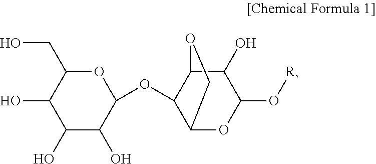
in Chemical Formula 1,
R is a C₁₋₅ alkyl group.

2. The compound of claim 1, wherein R is an ethyl group.

3. A method of producing a compound represented by Chemical Formula 1 below, the method comprising:
treating agar or agarose with a strong acid and an alkanol;
neutralizing a product from the treating process; and
separating and purifying a compound represented by Chemical Formula 1 below from the neutralized product: 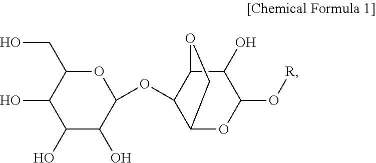
in Chemical Formula 1,
R is a C₁₋₅ alkyl group.

4. The method of claim 3, wherein the strong acid is one or more of hydrochloric acid, sulfuric acid, and nitric acid.

5. The method of claim 3, wherein 1 or 20% (w/v) of agar or agarose is treated with the strong acid at a concentration of 10 to 100 mM.

6. The method of claim 3, wherein the strong acid and the alkanol are treated sequentially or simultaneously, regardless of order.

7. The method of claim 3, wherein the alkanol is ethanol, and R is an ethyl group.

8. A moisturizing composition comprising a compound represented by Chemical Formula 1 below, a derivative thereof, or a salt thereof: 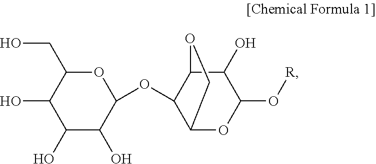
in Chemical Formula 1,
R is a C₁₋₅ alkyl group.

9. A moisturizing cosmetic composition comprising a compound represented by Chemical Formula 1 below, a derivative thereof, or a salt thereof: 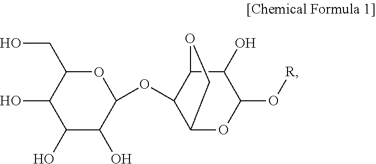
in Chemical Formula 1,
R is H or a C₁₋₅ alkyl group.

10. The composition of claim 8, wherein the alkyl group is an ethyl group.

11. The composition of claim 9, wherein the alkyl group is an ethyl group.
